(19) United States
(12) Patent Application Publication
(10) Pub. No.: US 2012/0142107 A1
(43) Pub. Date: Jun. 7, 2012

(54) METHOD AND SYSTEM FOR SOMATIC CELL NUCLEAR TRANSFER

(75) Inventor: Benjamin Bartoov, Moshav Hemed (IL)

(73) Assignee: MAZOREL LTD., Givat Shmuel (IL)

(21) Appl. No.: 13/321,672

(22) PCT Filed: May 20, 2010
(86) PCT No.: PCT/IL10/00402

§ 371 Date: Feb. 21, 2012

Related U.S. Application Data
(60) Provisional application No. 61/213,229, filed on May 19, 2009.

(51) Int. Cl. C12N 15/873; C12N 5/076; C12N 5/10

(52) U.S. Cl. 435/455; 435/325

(57) ABSTRACT

Provided is a method for nuclear transfer. An exogenous donor nucleus is introduced into an enucleated oocyte and one or more enucleated sperm cells or one or more enucleate sperm cell fractions are introduced into the oocyte. The one or more sperm cells or one or more sperm cell fractions may be introduced into the oocyte either before, after, or simultaneously, with the donor nucleus. Also provided are cells and embryos produced by the method.

### BACKGROUND OF THE INVENTION

The following prior art references are considered to be relevant for an understanding of the invention:

1. Wilmut I, Schnieke A E, McWhir J, Kind A J, Campbell K H. Viable offspring derived from fetal and adult mammalian cells. Nature. 1997 Feb. 27;385(6619):810-3. Erratum in: Nature 1997 Mar 13; 386(6621):200.

2. Cibelli J B, Lanza R P, West M D, Ezzel C. The first Human cloned Embryo, Scientific American, Nov. 24, 2001.

3. Stojkovic M, Stojkovic P, Leary C, Hall V J, Armstrong L, Herbert M, Nesbitt M, Lako M, Murdoch A. Derivation of a human blastocyst after heterologous nuclear transfer to donated oocytes. Reprod Biomed Online. 2005 August;11(2):226-31.

4. Heindryckx B, De Sutter P, Gerris J, Dhont M, Van der Elst J. Embryo development after successful somatic cell nuclear transfer to in vitro matured human germinal vesicle oocytes. Hum Reprod. 2007 July; 22(7):1982-90. Epub 2007 May 18.

5. French A J, Adams C A, Anderson L S, Kitchen J R, Hughes M R, Wood S H. Development of human cloned blastocysts following somatic cell nuclear transfer with adult fibroblasts. Stem Cells. 2008 February; 26(2):485-93. Epub 2008 Jan 17.

6. Jianyuan Li, Xuexia Liu, Haiyan Wang, Shouxin Zhang, Fujun Liu, Xuebo Wang, and Yanwei Wang. Human embryos derived by somatic cell nuclear transfer using an alternative enucleation approach. Cloning and stem cells. Volume 11 Number 1 2009.

7. Knott J G, Poothapillai K, Wu H, He C L, Fissore R A, Robl J M. Porcine sperm factor supports activation and development of bovine nuclear transfer embryos. Biol Reprod. 2002 April; 66(4):1095-103.

Stem cells have unique features that distinguish them from other cell types. They are capable of dividing and renewing themselves for long periods, they are unspecialized, and they can give rise to specialized cell types. There are several types of stem cells: embryonic stem cells, adult stem cells, umbilical cord blood stem cells, which are considered as highly plastic adult stem cells, placental stem cells and amniotic stem cells. Adult stem cells are undifferentiated cells found among differentiated cells in tissues and organs. These cells can differentiate to yield the major specialized cell types of the tissue or organ in which they are found and by this they maintain and repair the tissue in which they are found. Adult stem cells have been identified in many organs and tissues, but there are only a very small number of stem cells in each tissue. Embryonic stem cells are obtained from the inner cell mass of blastocysts, which are very early stage embryos, four to five days old, containing about 100 cells. These cells can give rise to each of the three founding germ layers of an early embryo: ectoderm, endoderm and mesoderm. Thus, these cells can differentiate into all of the cell types of the adult body when given appropriate stimulation.

Human stem cells pose great promise for clinical treatment of humans. These cells can be used for the generation of cells, tissues and organs that could be used in the field of medicine known as regenerative medicine. Organs created from human stem cells can become the source of transplanted artificial organs while cells and tissues produced from stem cells may be transplanted into humans in order to repair tissue damage caused by trauma, disease or aging. This renewable source of replacement cells and tissues may some day treat diseases including Parkinson's and Alzheimer's disease, spinal cord injury, stroke, burns, heart diseases, diabetes, osteoarthritis, and rheumatoid arthritis.

When considering the use of stem cells in regenerative medicine, embryonic stem cells have advantages over adult stem cells. The major advantage is the ability to easily obtain a large number of embryonic stem cells, while adult stem cells are rare in mature tissues and methods for expanding their number are not fully developed. On the other hand, while adult stem cells can originate from the patient's own body (autologous), embryonic stem cells are typically genetically different from the cells of the recipient (allogenic) and thus might be rejected by the recipient's immune system. In order to avoid this problem it is possible to use a technique that produces embryonic stem cells with the patient's own genetic material. This technique is called somatic cell nuclear transfer (SCNT). In SCNT the nuclear genetic material is removed from an oocyte and replaced with the genetic material from a patient's somatic donor cell. The oocyte is then induced to develop into an embryo until the blastocyst stage from which embryonic stem cells are harvested. These embryonic stem cells match the patient's cells and can be induced to undergo differentiation into a desired cell type as required.

In addition to its use in regenerative medicine, SCNT would have an important role in basic and clinical scientific studies. Stem cells derived from SCNT have an advantage of being genetically matched to the donor organism, thus enabling the researcher to use genetically-customized stem cells for his research. An example of this would be SCNT using a donor cell from a person that is suffering from a certain disease and thus creating stem cells that carry genes that are linked to the disease. These disease-specific stem cells would be studied in order to better understand the disease. Another example is the use of SCNT derived stem cells that carry relevant genes to test new drugs.

An additional important use of SCNT in animals is for reproductive cloning. Reproductive cloning is a technology used to generate an animal that has the same nuclear DNA as another currently or previously existing animal, generating genetically identical animals. An example for the use of reproductive cloning is the famous creation of Dolly the sheep (1). In this process an embryo is created in vitro using SCNT and when the cloned embryo reaches a suitable stage, it is transferred to the uterus of a female host where it continues to develop until birth. Reproductive cloning can be used to develop efficient ways to reproduce animals with special qualities, drug producing animals or animals that have been genetically altered to serve as models for studying human disease. Reproductive cloning also could be used to repopulate endangered animals or animals that are difficult to breed. Hundreds of cloned animals exist today, but the number of different species is limited.

The process of SCNT is highly inefficient and improvements in the technology are needed. Many animal species have not been able to be cloned yet and in animal species that are successfully cloned only about 20% of nuclear transfer procedures produce blastocysts. In the case of reproductive cloning, only about 1% of blastocysts lead to live births and many of the cloned animals born alive die young or are abnormal. In humans, the success rate of SCNT is much more limited. In 2001 the biotechnology company Advanced Cell Technology reported the production of the first human embryo produced by SCNT. This embryo reached only a four- to six-cell stage and could not further develop (2). In 2005 a group from the UK reported success in obtaining a human blastocyst following nuclear transfer using an undifferentiated embryonic stem cell as a donor cell but not an adult donor cell (3). In 2007, a Belgian group reported the production of the first SCNT derived human embryos using in-vitro matured oocytes but these embryos did not reached the blastocyst stage (4). An improvement was achieved at the beginning of 2008, when Stemagen Corporation from the USA reported the formation of blastocysts following nuclear transfer using adult skin fibroblast cells (5). In 2009, a Chinese group reported the achievement of similar results (6). If embryonic stem cells were produced from these blastocysts, they were autologous to the donor cells. Presently, no human stem cell lines were reported to be produced from SCNT.

An attempt was made to improve the success rate of SCNT by injection into the oocyte of a cytosolic fraction of porcine sperm cells referred to as “porcine sperm factor (pSF)” (7). However, injection of pSF into the oocyte following donor genome insertion, did not improve embryonic development following SCNT.

Somatic cell nuclear transfer is usually carried out as follows. Matured metaphase II (MII) oocytes with a visible polar body are transferred into cytochalasin B. The genetic material of the oocyte is made visible either by staining the DNA itself with a special dye (such as Hoechst) or by using polarized light microscopy that makes the metaphase plate (spindle) visible. A small hole is drilled in the zona pellucida using laser radiation and the oocytes are then enucleated by aspirating the metaphase plate, in a small amount of cytoplasm, using a micropipette that was inserted through the hole. Through the same hole in the zona pellucida, a donor cell (a whole cell or a cell whose membrane was damaged) is either injected into the oocyte or placed under the zona pellucida (in the perivitelline space) in close proximity to the plasma membrane. In the later case, fusion between the oocyte and the donor cell is induced by electrical pulses. A variety of stimuli are then applied in order to stimulate cell division and embryo formation. With a low frequency, the newly constructed cell will divide, replicating the donor cell DNA while remaining in a pluripotent state and an embryo will be formed.

### SUMMARY OF THE INVENTION

The present invention relates to methods and systems for somatic cell nuclear transfer. The invention may be used, for example, to clone an animal or to produce stem cells or differentiated cells containing the genetic information or the nucleic acid sequence of a donor nucleus. The donor nucleus and the oocyte may originate from the same individual organism, or from different individuals. The donor nucleus and the oocyte may be from the same species or from different species.

In accordance with the invention, one or more sperm cells or one or more sperm cell fractions are introduced into the oocyte, either before, after, or simultaneously with the donor nucleus. The inventors have found that introduction of sperm cells or sperm cell fractions into oocytes in a SCNT procedure improved SCNT outcomes by increasing the number of embryo cleavage days and the number of cells per embryo in comparison to control SCNT procedures in which sperm cells or sperm cell fractions were not introduced into the oocytes.

In embodiments of the invention in which one or more sperm cells are used, the sperm cells are enucleated sperm cells. An enucleated sperm cell is devoid of a nuclear genome, or is “functionally enucleated.” A functionally enucleated sperm cell contains a nuclear genome that is non-functional, e.g., cannot replicate or synthesize DNA. In one embodiment, one or more naturally occurring enucleated sperm cells are used. For example, naturally occurring headless sperm cells (sometimes referred to as “pin heads”) can be used. Alternatively, the sperm cells can be functionally enucleated, for example, by irradiation, by x-ray irradiation, by laser irradiation, by physically removing the genome, or by chemical means. The one or more of the sperm cells may initially be living sperm cells having normal motility or impaired motility or dead sperm cells.

Enucleated sperm cell fractions for use in the invention, are sperm cell fractions which are devoid of a nuclear genome, or in which the nuclear genome is “functionally enucleated”. Such sperm cell fractions suitable for use in the invention include for example:
(a) a naturally occurring headless sperm cell or a sperm cell from which the head was removed;
(b) a sperm cell in which the nucleus was damaged or removed;
(c) a sperm cell fraction comprising at least a portion of a sperm cell plasma membrane;
(d) a sperm cell fraction comprising the sperm cell middle piece;
(e) a sperm cell fraction comprising the sperm cell capitulum;
(f) a sperm cell fraction comprising at least a portion of an axoneme;
(g) a sperm cell fraction comprising mitochondrial sheath made of one or more mitochondria; and
(h) a sperm cell fraction comprising at least a portion of a sperm cell mitochondrial membrane;

The sperm cell(s) or sperm cell fraction(s) may be introduced into the oocyte by any method known in the art. For example, the sperm cell or sperm cell fraction may be introduced into the oocyte by intracytoplasmic sperm injection (ICSI). ICSI is a common in vitro fertilization procedure in which a sperm cell is injected directly into an egg, and may be used to introduce a sperm cell or sperm cell fragment into the oocyte. ICSI is carried out under a microscope using multiple micromanipulation devices (micromanipulators, microinjectors and micropipettes). A holding pipette is used to stabilize the oocyte. From the opposite site, a thin, hollow needle loaded with the sperm cell(s) or sperm cell fraction(s) to be inserted into the oocyte, is pierced through the oocyte membrane (the oolemma) and into the inner part of the oocyte. The sperm cell(s) or sperm cell fraction(s) are then injected from the needle into the oocyte. Alternatively, the sperm cell or sperm cell fraction may be introduced into the oocyte fusion of the sperm membrane with the oocyte membrane.

Prior to introduction of the sperm cell(s) or sperm cell fraction(s) into the oocyte, the tail of the sperm cell is preferably, but not necessarily, damaged and immobilized by squeezing it at its principle piece, for example, between the glass pipette and the bottom of the dish as performed routinely in ICSI procedures.

The donor cell can be any type of diploid cell, such as a somatic cell, gametocyte, or a stem cell. The term “somatic cell” as used herein refers to a differentiated cell. The cell can be a somatic cell or a cell that is committed to a somatic cell lineage. The donor cell can originate from an organism such as a human or an animal or from a cell and/or tissue culture system. If taken from a human or animal, the donor individual can be at any stage of development, for example, an embryo, a fetus or an adult. Additionally, the present invention can utilize embryonic cells. Embryonic cells can include embryonic stem cells as well as embryonic cells committed to a somatic cell lineage. Such cells can be obtained from the endoderm, mesoderm or ectoderm of the embryo. Embryonic cells committed to a somatic cell lineage are usually isolated on or after approximately day ten of embryogenesis. However, cells can be obtained prior to day ten of embryogenesis. If a cell line is used as a source for a chromosomal genome, then primary cells are preferred. The term “primary cell line” as used herein includes primary cells as well as primary derived cell lines.

Suitable somatic cells for use as a donor cell include fibroblasts (for example, primary fibroblasts), epithelial cells, muscle cells, cumulus cells, neural cells, sertoli cells, and mammary cells. Other suitable cells include hepatocytes and pancreatic islet cells.

Somatic cells can be obtained, for example, by disassociation of tissue by mechanical (e.g., chopping, mincing) or enzymatic (e.g., trypsinization) means to obtain a cell suspension followed by culturing the cells until a confluent monolayer is obtained. The somatic cells can then be harvested and prepared for cryopreservation, or maintained as a stock culture.

The donor cell from which the donor nucleus is obtained is preferably in the active G₁ phase of its cell cycle (engaged in the mitotic cell cycle) or not active and arrested in the G₀ phase of its cell cycle (a resting stage of mitosis). G₁ phase cells can be obtained from an actively dividing cell culture, while G₀ phase cells can be obtained by incubation of the cells in a serum-free starvation medium. The donor cell may also be activated in the S phase or G₂/M phase, which is the transitional phase between the G₂ and the M phase.

The genome of the donor cell can be the naturally occurring genome, for example, for the production of cloned animals or cells, or the genome can be genetically altered to comprise a transgenic sequence, for example, for the production of transgenic cloned animals.

The oocytes used in the present invention may be in the second metaphase (MII) stage of meiotic cell division. Oocytes in metaphase II are considered to be in a resting state. The stage of the oocyte can be identified by visual inspection of the oocyte. Oocytes that are in metaphase II are identified, for example, by the presence of the first polar body. Methods for identifying the stage of meiotic cell division are known in the art.

The oocytes used in the present invention may be activated oocytes. Activated oocytes are those that are in a dividing stage of meiotic cell division, and include metaphase I, anaphase I, anaphase II or telophase II. Methods for activating an oocyte are known in the art.

Oocytes can be obtained from a donor organism at various stages of the organism's reproductive cycle. For example, oocytes can be aspirated from follicles of ovaries at various times during the reproductive cycle. Around the time of ovulation, some oocytes may be obtained in metaphase II. Oocytes obtained in other stages may be induced to enter metaphase II in-vitro (in-vitro maturation) using in-vitro hormonal stimulation. Alternatively, oocytes can be collected from a female following exogenous hormone-stimulation leading to super ovulation. Methods of in-vitro maturation, induction of super ovulations and collection of the oocytes are known in the art.

An enucleated oocyte is one that is devoid of a nuclear genome, or one that is “functionally enucleated.” A functionally enucleated oocyte contains a genome that is non-functional, e.g., cannot replicate or synthesize DNA. The oocyte can be functionally enucleated, for example, by irradiation, by x-ray irradiation, by laser irradiation, by physically removing the genome, or by chemical means. More preferably, however, the nuclear genome of the oocyte is physically removed from the oocyte.

To physically remove the nuclear genome of an oocyte, the genetic material of the oocyte is made visible either by staining the DNA itself using a dye that stains DNA (such as Hoechst) or by using polarized light microscopy that makes the spindle apparatus (metaphase plate) visible. A small hole can be generated in the zona pellucida using laser radiation or by cutting the zone pellucida with a micropipette. Then one can insert a micropipette through the hole, enter the plasma membrane of the oocyte and remove the nuclear material from the oocyte in a small amount of cytoplasm.

The oocyte can be functionally enucleated also by chemical methods. Methods of chemically inactivating the DNA are known to those of skill in the art. For example, chemical inactivation can be performed using the etoposide-cycloheximide method. The genome of an oocyte can be inactivated by treating the oocyte with a compound that induces the oocyte nuclear genome to segregate into the polar bodies during meiotic maturation thereby leaving the oocyte devoid of a functional genome, and resulting in the formation of a recipient cytoplast for use in nuclear transfer procedures. Examples of agents that effect such differential segregation include agents that disrupt the cytoskeletal structures include Taxol (e.g., paclitaxel), demecolcine, phalloidin, colchicine, nocodozole. Examples of agents that effect such differential segregation also include agents that disrupt metabolism such as cycloheximide and tunicamycin. In addition, exposure of oocytes to other agents or conditions (e.g. increased or decreased temperature, pH, osmolarity) that preferentially induce the skewed segregation of the oocyte genome so as to be extruded from the confines of the oocyte (e.g., in polar bodies) are also known.

Inserting the nuclear genome from the donor cell into the enucleated oocyte can be carried out in any one of a variety of ways. The genome of a donor cell can be injected into the oocyte by employing a microinjector (i.e., micropipette or needle). The nuclear genome of the donor cell, for example, a somatic cell, can be extracted using a micropipette or needle. Once extracted, the donor nuclear genome can be placed in the activated oocyte by inserting the micropipette, or needle, into the oocyte and releasing the nuclear genome of the donor cell into the cytoplasm of the oocyte.

The genome of a donor cell can also be inserted into an oocyte by fusion e.g., electrofusion, viral fusion, liposomal fusion, biochemical reagent fusion (e.g., phytoheniaglutinin (PHA) protein), or chemical fusion (e.g., polyethylene glycol (PEG) or ethanol). The nucleus of the donor cell can be deposited beneath the zona pelliduca surrounding the oocyte, in the perivitelline space. Fusing the donor nucleus with the oocyte can then be performed by applying an electric field which also results in activation of the oocyte.

For generating transgenic cells or organisms, a donor nucleus is used from a donor cell with a genetically engineered genome. Such a combination results in a transgenic nuclear transfer embryo. A transgenic organism is an organism that has been produced from a genome from a donor cell that has been genetically altered, for example, to produce a desired protein. Methods for introducing DNA constructs into the germ line of an animal to make a transgenic animal are known in the art. Genetically engineered donor cells for use in the invention can be obtained from a cell line into which a nucleic acid of interest, for example, a nucleic acid which encodes a protein, has been introduced.

Following insertion of the donor nuclear genome into the oocyte, the oocyte should undergo activation in order to stimulate cell division and embryonic development. The activation can be performed in a variety of ways. Activation can be achieved by electrical stimulation or by exposure to elevated extracellular calcium, possibly together with a calcium ionophore, such as ionomycin or A23187, and/or by exposure to ethanol. Activation can also be achieved by inhibition of phosphorylation in the oocyte, for example using 6-dimethylaminopurine (6-DMAP) or by inhibition of protein synthesis in the oocyte, for example using cycloheximide. Activation can be further enhanced by injection of a variety of substances into the oocyte. A combination of several treatments may be performed.

In its first aspect, the present invention provides method for somatic cell nuclear transfer comprising introducing into an oocyte one or more enucleated sperm cells or one or more enucleated sperm cell fractions.

The one or more of the sperm cells may be a morphologically normal sperm cell, a living sperm cell, a dead sperm cell, a naturally occurring headless sperm cell, a sperm cell having impaired motility, a motile sperm cell.

The sperm cell tail of the enucleated sperm cells or sperm cell fractions may be damaged and immobilized by squeezing it at its principle piece, between the glass pipette and the bottom of the dish.

The one or more sperm cell fractions may be selected from
(a) a naturally occurring headless sperm cell or a sperm cell from which the head was removed;
(b) a sperm cell in which the nucleus was damaged or removed;
(c) a sperm cell fraction comprising at least a portion of a sperm cell plasma membrane;
(d) a sperm cell fraction comprising the sperm cell middle piece;
(e) a sperm cell fraction comprising the sperm cell capitulum;
(f) a sperm cell fraction comprising at least a portion of an axoneme;
(g) a sperm cell fraction comprising mitochondrial sheath made of one or more mitochondria; and
(h) a sperm cell fraction comprising at least a portion of a sperm cell mitochondrial membrane;

The one or more sperm cells or one or more sperm cell fractions are introduced into the oocyte either before, after, or simultaneously with the donor nucleus.

The one or more sperm cells or one or more sperm cell fractions are introduced into the oocyte by intracytoplasmic sperm injection.

In its second aspect, the present invention provides a system for somatic cell nuclear transfer comprising:
(a) an enucleated oocyte; and
(b) one or more enucleated sperm cells or one or more enucleate sperm cell fractions.

Thus, in one of its aspects, the present invention provides a method for nuclear transfer comprising:
(a) introducing into an enucleated oocyte an exogenous nucleus; and
(b) introducing into the oocyte one or more enucleated sperm cells or one or more enucleate sperm cell fractions.

In the method of the method one or more of the enucleated sperm cells may be produced from a morphologically normal sperm cell, a living sperm cell, a dead sperm cell, a naturally occurring headless sperm cell, a sperm cell having impaired motility, a motile sperm cell. The sperm cell tail of the enucleated sperm cells or sperm cell fractions may be damaged and immobilized by squeezing it at its principle piece, between the glass pipette and the bottom of the dish. The one or more sperm cell fractions are selected from:
(a) a naturally occurring headless sperm cell or a sperm cell from which the head was removed;
(b) a sperm cell in which the nucleus was damaged or removed;
(c) a sperm cell fraction comprising at least a portion of a sperm cell plasma membrane;
(d) a sperm cell fraction comprising the sperm cell middle piece;
(e) a sperm cell fraction comprising the sperm cell capitulum;
(f) a sperm cell fraction comprising at least a portion of an axoneme;
(g) a sperm cell fraction comprising mitochondrial sheath made of one or more mitochondria; and
(h) a sperm cell fraction comprising at least a portion of a sperm cell mitochondrial membrane;

Furthermore, in the method of the invention, the one or more sperm cells or one or more sperm cell fractions may be introduced into the oocyte either before, after, or simultaneously with the donor nucleus. The one or more sperm cells or one or more sperm cell fractions may be introduced into the oocyte by intracytoplasmic sperm injection or by fusing a sperm membrane with an oocyte membrane.

In another of its aspects, the invention provides a system for nuclear transfer comprising:
(a) an enucleated oocyte; and
(b) one or more enucleated sperm cells or one or more enucleate sperm cell fractions.

The invention also provides a cell produced by the method of the invention and an embryo produced by the method of the invention.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a histogram showing the number of cells per embryo following somatic cell nuclear transfer, with and without injection of sperm cell tail/s, using germinal vesicle (GV) oocytes that matured in-vitro to become metaphase II (MII) oocytes.

FIG. 2 is a histogram showing the number of embryo cleavage days following somatic cell nuclear transfer, with and without injection of sperm cell tail/s, using germinal vesicle (GV) oocytes that matured in-vitro to become metaphase II (MII) oocytes.

FIG. 3 is a histogram showing the number of cells per embryo following somatic cell nuclear transfer, with and without injection of sperm cell tail/s, using in-vivo matured metaphase II (MII) oocytes.

FIG. 4 is a histogram showing the number of embryo cleavage days following somatic cell nuclear transfer, with and without injection of sperm cell tail/s, using in-vivo matured metaphase II (MII) oocytes.

### EXAMPLES

Materials

Activation Medium:

0.3 M Manitol, 0.1 mM MgSO₄, 50 μM CaCl₂

Methods

Somatic Cell Nuclear Transfer

Preparation of Cytoplasts-Oocyte Enucleation:

Cumulus-free MII oocytes (that either matured in-vivo or GVs that matured in-vitro) with a visible first polar body were incubated in 5 μg/ml cytochalasin B in HTF-HEPES medium in a glass bottom plate for 30 min. During the incubation, the spindle apparatus (metaphase plate) of the oocytes was visualized using polarized light (LC-PolScope system of Cri Company). When the spindle was visible, a small hole was created in the zona pellucida near the spindle, using laser radiation. In cases where the polar body was far from the spindle, two holes were generated in the zona, one close to the polar body and the other close to the spindle. The spindle and the first polar body were aspirated from the oocyte using a blunt glass pipette OD 15 μm inserted through the hole in the zona pellucida.

Somatic Cell Nuclear Transfer:

Cells chosen for donor karyoplasts were fibroblast cells from actively dividing primary cultures of foreskin cells. The donor cells, placed in 5 cm plates, were trypsinized (0.5 ml of 0.25% Trypsin, 3-4 min. incubation at 37° C.) and resuspended in 3 ml DMEM medium. Donor cell suspension (18 μl) was mixed with 2 μl of 10× lectin (final concentration 300 μg/ml) and the cells were incubated at room temperature until use. The cells chosen for donor karyoplasts were small (10-15 μm in diameter) and most probably in G1 phase. A donor cell surrounded with lectin was inserted through the hole in the zona pellucida into the perivitelline space and attached to the oocyte plasma membrane. When sperm cell tails were used, they were severed from normal sperm cells using an ICSI glass pipette OD 7 μm or naturally occurring “Pin-head” sperm cells were isolated from a semen sample. The tails were squeezed at the principle piece, between the glass pipette and the bottom of the dish, as performed routinely in ICSI procedures. This squeezing was performed beneath the mitochondria to avoid damaging the mitochondria. Immediately after sperm tail preparation, the sperm tail was injected into the oocyte using an ICSI glass pipette (OD 7 μm). The oocyte-donor cell couples were transferred through three drops, the first contained HTF-HEPES: activation medium 3:1, the second contained HTF-HEPES: activation medium 1:1 and the third contained HTF-HEPES: activation medium 1:3. Then oocyte-donor cell couples were placed in 100% activation medium in a micro-fusion chamber between the two electrodes and the following electrical pulses were applied: one pulse of 0.17 kV cm ⁻¹ (3.4 V in the system used) for 5 seconds and two pulsed of 1.7 kV cm⁻¹ (34 V) for 15 μs with 0.1 second interval between the two pulses. The resultant SCNT reconstructions were transferred to Sage cleavage medium and incubated for 1 hour in the incubator. During the incubation period fusion was validated.

Chemical Activation:

The SCNT reconstructions were incubated in 10 μM calcium ionophore A-23187 in Sage cleavage medium for 4 min in the incubator. Then they were washed three times in fresh Sage cleavage medium and incubated in Sega cleavage medium supplemented with 2 mM 6-dimethylaminopurine (6-DMAP) for 1.5-4 hours in the incubator (under paraffin oil).

Embryo Culture:

Following the incubation with 6-DMAP, “embryos” were washed thoroughly with Sage cleavage medium and cultured in cleavage medium under paraffin oil in the incubator. On late day 3 or early day 4, embryos were transferred to Sage Blastocyst medium. The rate of embryonic development was evaluated throughout the in-vitro culture.

Results

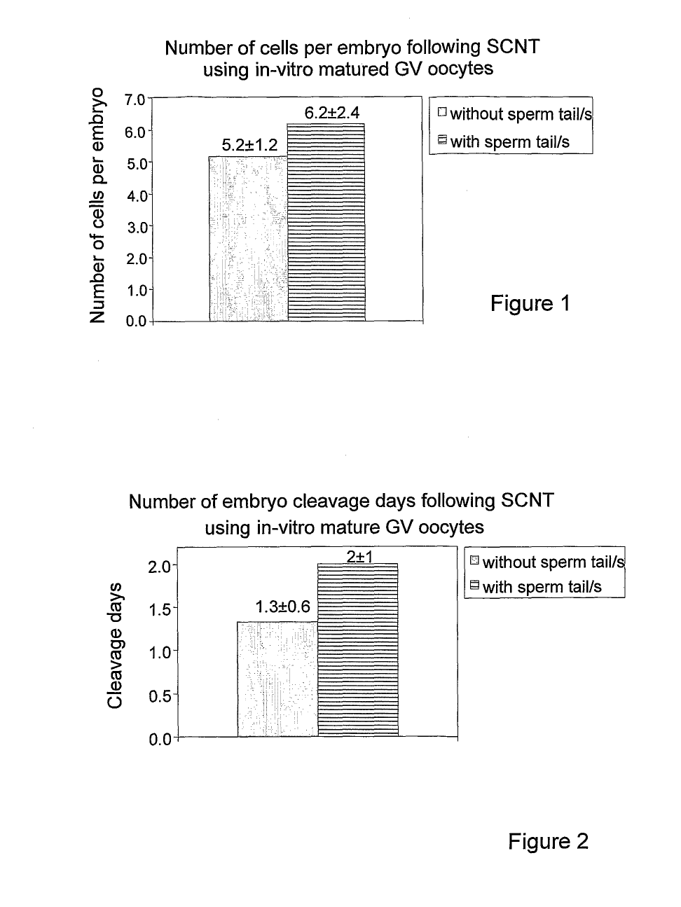

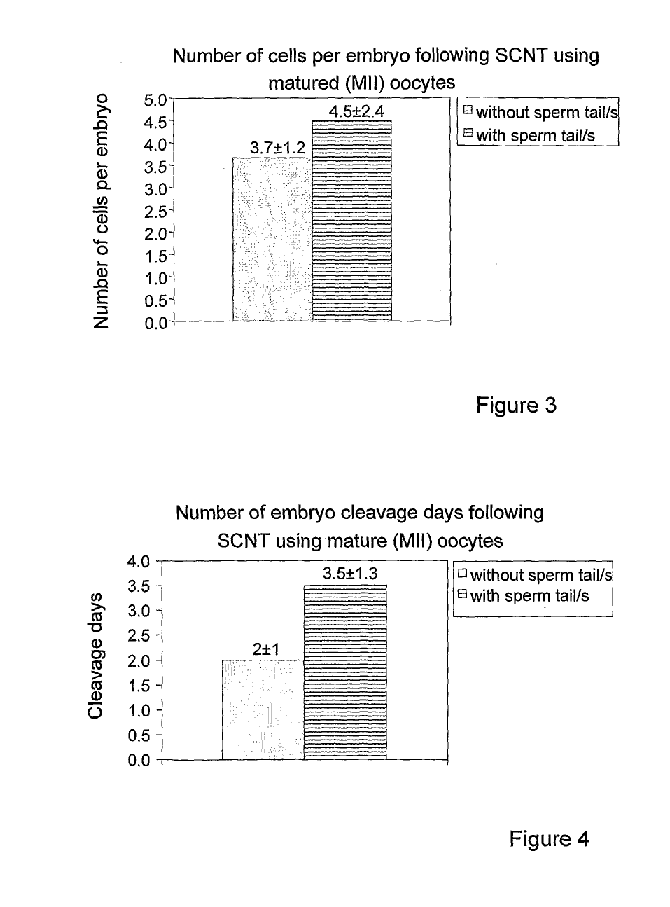

Somatic cell nuclear transfer was performed either with or without injection of human sperm tail/s into the oocyte. When human GV oocytes that matured in-vitro to become MII were used, injection of sperm tail/s into oocytes increased the number of cells per embryo by 19% (FIG. 1) and the number of embryo cleavage days by 54% (FIG. 2). When in-vivo matured (MII) human oocytes were used, injection of sperm tail/s into oocytes increased the number of cells per embryo by 22% (FIG. 3) and the number of embryo cleavage days by 75% (FIG. 4).

FIG. 1 shows the number of cells per embryo in SCNT using GV oocytes that matured in-vitro that were injected with sperm tail/s (right column) in comparison to controls (left column) which were not injected with sperm tail/s. Injection of sperm tail/s into the oocytes increased the number of cells per embryo by 19%. FIG. 2 shows that in these experiments the number of embryo cleavage days was 54% higher in the oocytes injected with sperm tail/s (right column) in comparison to the controls. When in-vivo matured (MII) human oocytes were used in SCNT, injection of sperm tail/s into oocytes increased the number of cells per embryo by 22% (FIG. 3) and increased the number of embryo cleavage days by 75% (FIG. 4).

1.-10. (canceled)

11. A method for nuclear transfer, comprising:
introducing into an enucleated oocyte an exogenous nucleus; and
introducing into the oocyte one or more sperm cells from which the head was removed or one or more naturally occurring headless sperm cells.

12. The method according to claim 11, wherein one or more of the sperm cells is produced from a morphologically normal sperm cell, a living sperm cell, a dead sperm cell, a sperm cell having impaired motility, and a motile sperm cell.

13. The method according to claim 11, wherein the at least one sperm cell of the one or more sperm cells has a tail that is damaged and immobilized by squeezing it at its principle piece, between the glass pipette and the bottom of the dish.

14. The method according to claim 11, wherein the one or more sperm cells are introduced into the oocyte either before, after, or simultaneously with the exogenous nucleus.

15. The method according to claim 11, wherein the one or more sperm cells are introduced into the oocyte by intracytoplasmic sperm injection.

16. The method according to claim 11, wherein the one or more sperm cells are introduced into the oocyte by fusing a sperm membrane with an oocyte membrane.

17. A system for nuclear transfer, comprising:
an enucleated oocyte; and
one or more sperm cells from which the head was removed or one or more naturally occurring headless sperm cells.

18. A cell produced by the method of claim 11.

19. An embryo produced by the method of claim 11.
